(19) United States
(12) Patent Application Publication
(10) Pub. No.: US 2024/0277237 A1
(43) Pub. Date: Aug. 22, 2024

(54) PULSE CONDITION PREDICTION METHOD AND SYSTEM

(71) Applicant: WISTRON CORP., New Taipei City (TW)

(72) Inventor: CHIN SHUO KUO, New Taipei City (TW)

(21) Appl. No.: 18/317,959

(22) Filed: May 16, 2023

(30) Foreign Application Priority Data
Feb. 22, 2023 (TW) 112106452

(51) Int. Cl. A61B 5/021; A61B 5/00

(52) U.S. Cl. CPC A61B 5/02108; A61B 5/7267

(57) ABSTRACT

A pulse condition prediction method and system. The pulse condition prediction system includes a pressure sensing module and a processing module. The pulse condition prediction method includes: sensing, by a pressure sensing module, an artery of a first subject to obtain a first arterial waveform, generating, by a processing module, to-be-predicted data basing on the first arterial waveform, wherein the to-be-predicted data includes pieces of first pulse wave data of meridians, and inputting, by the processing module, into a pulse condition prediction model, and predicted probability values of pulse conditions being generated by the pulse condition prediction model. Accordingly, pulse condition prediction result with high accuracy may be generated. Chinese medicine practitioner may perform more accurate and efficient diagnosis on the subject's health condition according to predicted probability values of the pulse conditions generated by the pulse condition prediction model and other determination results of look, listen, question and/or feel.

### CROSS-REFERENCE TO RELATED APPLICATIONS

This non-provisional application claims priority under 35 U.S.C. § 119(a) on Patent Application No(s). 112106452 filed in Republic of China (ROC) on Feb. 22, 2023, the entire contents of which are hereby incorporated by reference.

### BACKGROUND

### 1. Technical Field

This disclosure relates to a pulse condition prediction method and system.

### 2. Related Art

In the past ten years, with the rapid development of traditional Chinese medicine, the principles of feeling the patient's pulses on meridians have gradually become well-known based on many scientific methods, and it is no longer a difficult medicine. Traditional Chinese medicine is performed through logical evaluation based on various information logic to evaluate the physical condition of patients. Said various information may be obtained by looking, listening, questioning and feeling, wherein “feeling” refers to taking the pulse by pressing on the arteries close to the wrist joint on the back of the thumb to observe the changes in the pulse. The pulse reflects not only the heartrate, autonomic nerves, endocrine, etc., can also directly affect the tightness and thickness of the arteries; whether normal or not of the function of the organs also affects the concentration of the content of the arteries.

Despite the current development of using pulse diagnosis machines for pulse diagnosis, considering only the positive and negative values of the “qi (amplitude)” for each meridian, there are 2 to the power of 11 (2048) possible variations, and the oxygen deficiency index of the qi also has positive and negative values (2048); the “blood (phase)” also has positive and negative values (2048), and the oxygen deficiency index of the blood also has positive and negative values (2048). Therefore, just considering the qualitative data (positive or negative) alone, there are 2048×2048×2048×2048 possible variations. Even with the assistance of pulse diagnosis machines, it is still difficult for traditional Chinese medicine practitioners to make efficient and accurate diagnoses based on a large amount of data.

### SUMMARY

According to one or more embodiment of this disclosure, a pulse condition prediction method includes: sensing, by a pressure sensing module, an artery of a first subject and a first arterial waveform being obtained by the artery of the first subject; generating, by a processing module, to-be-predicted data basing on the first arterial waveform, wherein the to-be-predicted data comprises a plurality of pieces of first pulse wave data of a plurality of meridians; and inputting, by the processing module, the to-be-predicted data into a pulse condition prediction model, and a plurality of predicted probability values of a plurality of pulse conditions being generated by the pulse condition prediction model having the to-be-predicted data.

According to one or more embodiment of this disclosure, a pulse condition prediction system includes: a pressure sensing module and a processing module. The pressure sensing module is configured to sense an artery of a first subject and a first arterial waveform is generated by the pressure sensing module. The processing module is connected to the pressure sensing module, and is configured to generate to-be-predicted data via the first arterial waveform, and input to-be-predicted data into a pulse condition prediction model, and a plurality of predicted probability values of a plurality of pulse conditions are generated by the pulse condition prediction model having the to-be-predicted data, wherein the to-be-predicted data comprises a plurality of pieces of first pulse wave data of a plurality of meridians.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a block diagram illustrating a pulse condition prediction system according to an embodiment of the present disclosure;

FIG. 2 is a flow chart illustrating a pulse condition prediction method according to an embodiment of the present disclosure;

FIG. 3 is a schematic diagram showing locations of cun, guan and chi;

FIG. 4 is a schematic diagram showing diagnosis chart of pulse wave;

FIG. 5 is a schematic diagram showing waveforms corresponding to a phase difference tag;

FIG. 6 is a schematic diagram showing training data;

FIG. 7 is a classification structural diagram of an initial neural network model used in the present disclosure;

FIG. 8 is a block diagram illustrating a pulse condition prediction system according to another embodiment of the present disclosure;

FIG. 9 is a flow chart illustrating a pulse condition prediction method according to another embodiment of the present disclosure; and

FIG. 10 is a schematic diagram illustrating model training and model application according to one or more embodiments of the present disclosure.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the present invention. The following embodiments further illustrate various aspects of the present invention, but are not meant to limit the scope of the present invention.

Traditionally, relying on manually taking the pulse of a subject can only get the results of the 11 meridians of the left hand and the right hand respectively, wherein the location for taking the pulse includes upper section, middle section and lower section (cun, guan and chi) of the wrist. Further, said 11 meridians are divided into 22 areas, meaning that the precision of taking the pulse includes liver (2—middle/lower), kidney (2—middle/lower), spleen (2—middle/lower), lung (1—middle), stomach (3), gallbladder (3), bladder (3), large intestine (2—upeer/middle), triple warmer (2—upeer/middle) and small intestine (2—upeer/middle). One hand corresponds to 22 areas of the body, meaning that there are total of 44 results of taking the pulse. Based on the above, it can be known that traditional Chinese medicine practitioners can only diagnose a patient based on the result of taking the pulse by relying on their own experiences. Through the pulse condition prediction system and method of the present disclosure, traditional Chinese medicine practitioners can make efficient and accurate diagnoses on the subject's health condition according to predicted probability values generated by a pulse condition prediction model and other determination results obtained by the practitioners performing looking, listening, questioning and feeling. Therefore, misdiagnosis made by traditional Chinese medicine practitioners with less experience can be avoided, and the accuracy of diagnoses made by experienced traditional Chinese medicine practitioners can be further improved.

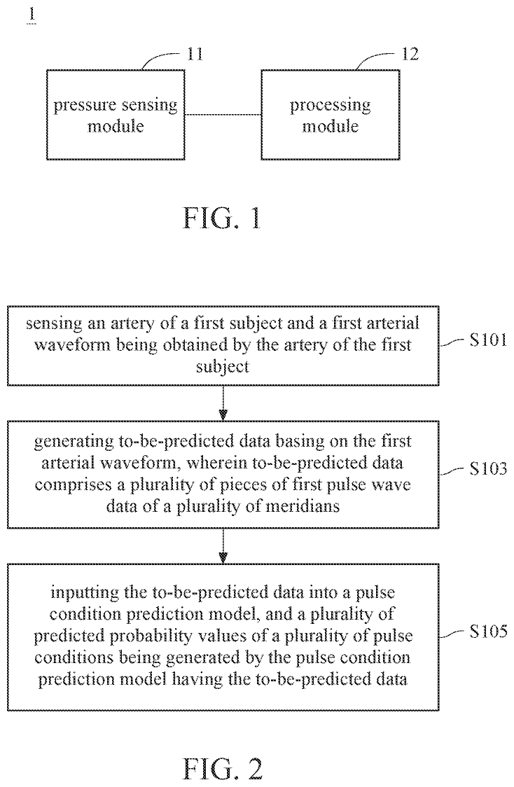

Please refer to FIG. 1, FIG. 1 is a block diagram illustrating a pulse condition prediction system according to an embodiment of the present disclosure. As shown in FIG. 1, the pulse condition prediction system 1 includes a pressure sensing module 11 and a processing module 12. The processing module 12 is connected to the pressure sensing module 11, wherein the processing module 12 may be electrically connected to the pressure sensing module 11 or in communication connection with the pressure sensing module 11.

The pressure sensing module 11 may be implemented in the form of a wristband for sensing artery in the wrist. In an implementation, the pressure sensing module 11 includes one or more sensors configured to sense artery of a first subject to generate a first arterial waveform; the processing module 12 includes one or more processors, and may be used to generate training data, train a model, and use the trained model (for prediction). Further, in the implementation of more than one processor, the processor configured to transform sensing data into training data and the pressure sensor may form a pulse diagnosis instrument (for example, the pulse diagnosis instrument developed by Professor Wang Wei-Kong). Said processor is, for example, a central processing unit (CPU), a graphics processing unit (GPU), a microcontroller, a programmable logic controller (PLC) or any other processors with signal processing function.

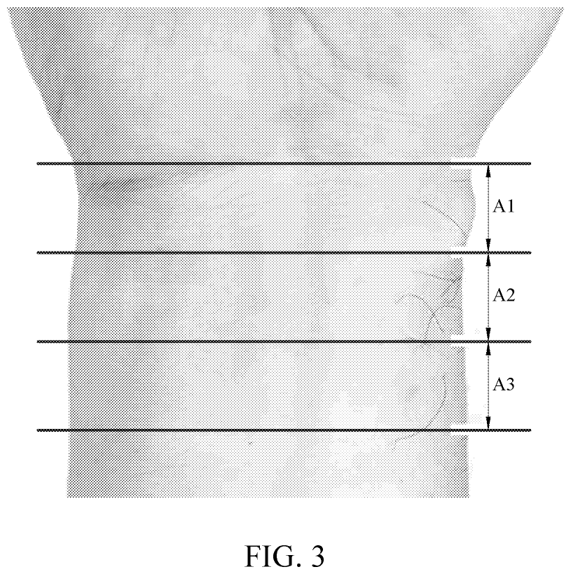

To explain the operation of the pulse condition prediction system 1 in more detail, please refer to FIG. 1, FIG. 2 and FIG. 3, wherein FIG. 2 is a flow chart illustrating a pulse condition prediction method according to an embodiment of the present disclosure, and FIG. 3 is a schematic diagram showing locations of cun, guan and chi. As shown in FIG. 2, the pulse condition prediction method includes: step S101: sensing an artery of a first subject and a first arterial waveform being obtained by the artery of the first subject; step S103: generating to-be-predicted data basing on the first arterial waveform, wherein the to-be-predicted data comprises a plurality of pieces of first pulse wave data of a plurality of meridians; and step S105: inputting the to-be-predicted data into a pulse condition prediction model, and a plurality of predicted probability values of a plurality of pulse conditions being generated by the pulse condition prediction model having the to-be-predicted data.

In step S101, the user or the processing module 12 activates the pressure sensing module 11 to sense the artery of the first subject and the first arterial waveform corresponding to the sensing result is obtained. Take FIG. 3 for example, the pressure sensing module 11 may be disposed on radial artery at the wrist of the first subject to measure the artery at a first section A1, a second section A2 and a third section A3. Further, the first section A1 may be the “cun” section in traditional Chinese medicine, which reflects the state of organs above the diaphragm; the second section A2 may be the “guan” section in traditional Chinese medicine, which reflects the state of organs in the abdominal area; the third section A3 may be the “chi” section in traditional Chinese medicine, which reflects the state of organs in the pelvic area. For example, in a normal pulse condition, cun, guan and chi have even thickness, if the result of taking the pulse indicates that “chi” section on the left wrist is thin and weak, it means that the subject's “Qi” in kidney is deficient, and there must be illness at the subject's pelvic cavity. Due to the asymmetry of the left and right organs of the human body, information obtained by taking/measuring the pulses on the “cun” sections of both hands is also different. The “cun” section is the “holographic unit” of the human body, which reflects the state of the entire body. However, the artery of the “cun” section is only in a narrow part between fingers, the amount of information can be obtained thereform depends on the skill and experience of the traditional Chinese medicine practitioners. Therefore, by actuating the pressure sensing module 11 to perform sensing, results with higher accuracy can be obtained.

In step S103, the processing module 12 generates the to-be-predicted data basing on the first arterial waveform, wherein the to-be-predicted data includes a plurality of pieces of first pulse wave data of a plurality of meridians. Specifically, each meridian has a corresponding resonant frequency, and the first arterial waveform is the waveform when the frequency of the heartbeat and the resonant frequency of the meridian are doubled frequency. In other words, a frequency of the pulse is a fundamental frequency, the wave of the meridian is the harmonic of the fundamental frequency, and because resonance is generated between the harmonic and the fundamental frequency, the first pulse wave data of each meridian may be measured based on resonance principle.

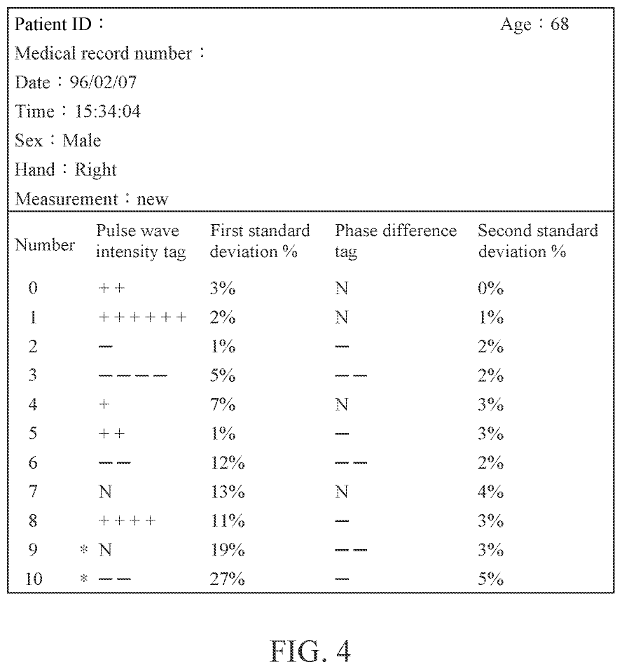

Please refer to FIG. 4, FIG. 4 is a schematic diagram showing diagnosis chart of pulse wave, wherein the diagnosis chart of FIG. 4 is obtained by the pulse diagnosis instrument developed by Professor Wang Wei-Kong. The pressure sensing module of the pulse diagnosis instrument is tied up at the locations of said cun, guan and chi. The diagnosis chart of pulse wave is obtained by recording five to six arterial waveforms and eliminating noise therefrom. The upper half of the diagnosis chart of pulse wave shows basic data of the first subject, and the lower half of the diagnosis chart of pulse wave shows numbers from 0 to 10, which represent the 11 meridians of heart, liver, kidney, spleen, lung, stomach, gallbladder, bladder, large intestine, triple warmer and small intestine. Each of the pieces of first pulse wave data may include a pulse wave intensity tag and a phase difference tag of a corresponding meridian, wherein the phase difference tag is associated with a phase difference between a phase of the first arterial waveform and a default phase. In the traditional Chinese medical science, the pulse wave intensity tag may indicate that “Qi” is strong or weak, and the phase difference tag may indicate that “Qi-blood” transformation is normal or not. In general, the impact of “blood” comes later than “Qi”, this is because variation of the corresponding phase difference of “blood” is only presented after the “qualitative change” of the organ, and “Qi” is just the amplitude (pulse amplitude) of the arterial waveform. Therefore, variations in “blood” are much more serious than variations in “Qi”. In short, the pulse wave intensity tag indicates that the heart of the first subject is strong or weak, and the phase difference tag indicates the phase difference between the phase of the first arterial waveform and the default phase of normal condition. The sign “+” in the column of the pulse wave intensity tag (intensity flag) indicates that the pulse wave is stronger than average, sign “−” in the column of the pulse wave intensity tag (intensity flag) indicates that the pulse wave is weaker than average, and sign “N” in the column of the pulse wave intensity tag (intensity flag) indicates that the pulse wave roughly equals to average, or that the pulse wave is between an upper threshold and a lower threshold of the intensity average of pulse wave. In addition, each pulse wave intensity tag may correspond to an amplitude standard deviation, such as a first standard deviation described below.

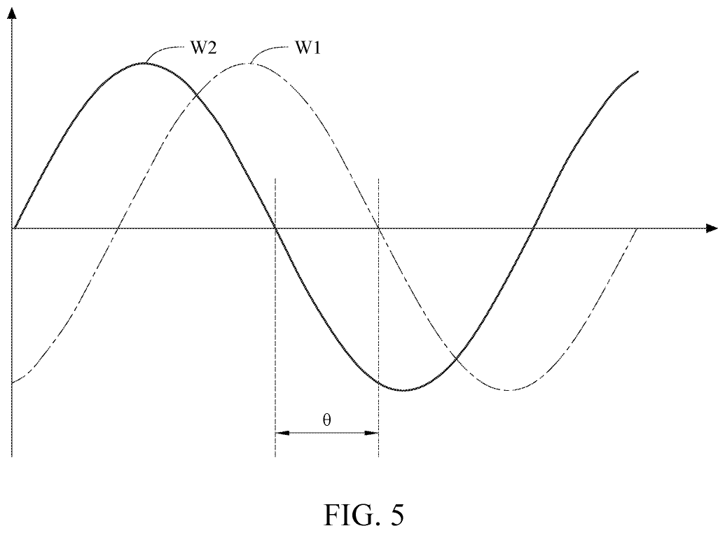

Please refer to FIG. 4 and FIG. 5, wherein FIG. 5 is a schematic diagram showing waveforms corresponding to the phase difference tag. The sign “−” in the column of the phase difference tag (phase flag) indicates that the phase difference θ between the phase of the first arterial waveform W1 and the default phase of the arterial waveform W2 of normal condition is higher than the average phase difference (for example, the phase of the first arterial waveform W1 lags behind the default phase of the arterial waveform W2 of normal condition by the phase difference θ), and the sign “N” in the column of the phase difference tag (phase flag) indicates that the phase difference θ between the phase of the first arterial waveform W1 and the default phase of the arterial waveform W2 of normal condition is roughly the same as the average phase difference, or is between an upper threshold and a lower threshold of the average phase difference. In addition, each phase difference tag may correspond to a phase standard deviation, such as a second standard deviation described below.

In addition, as shown in FIG. 4, each of the pieces of the first pulse wave data may further include the first standard deviation corresponding to the pulse wave intensity tag and the second standard deviation corresponding to the phase difference tag. Each of the meridians may have a corresponding default standard deviation. If the standard deviation of one of the meridians is higher than the default standard deviation, it means that said meridian might be abnormal. For example, the default standard deviation for the meridians numbered from 0 to 5 may be 7%, and the default standard deviation for the meridians numbered from 6 to 10 may be 15%, and therefore the meridians numbered 9 and 10 may be seen as abnormal (marked with the sign “*”).

In step S105, the processing module 12 inputs the to-be-predicted data into the pulse condition prediction model to generate the predicted probability values of the pulse conditions, wherein the pulse condition prediction model is a model trained prior to step S105. Said pulse conditions may include kidney asthenia, lung asthenia and anemofrigid cold etc., the present disclosure does not limit the content of the pulse conditions. Take the three pulse conditions listed above for example, the predicted probability values may include the predicted probability value of the first subject having kidney asthenia, the predicted probability value of the first subject having lung asthenia, and the predicted probability value of the first subject having anemofrigid cold. For example, assuming that the predicted probability values of kidney asthenia, lung asthenia and anemofrigid cold of a subject are 5%, 10% and 90% respectively, it means that the subject is likely to have anemofrigid cold.

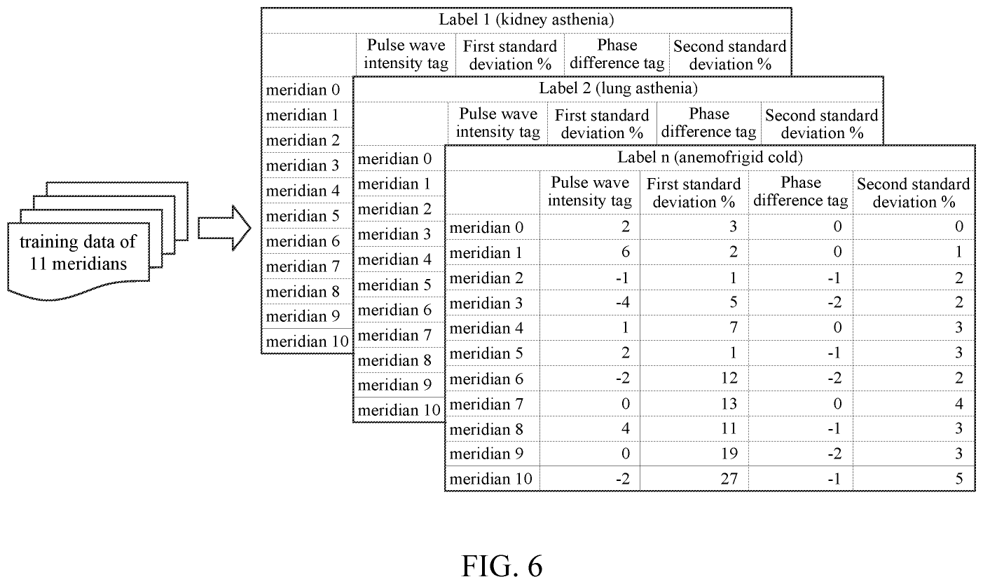

Please refer to FIG. 6, FIG. 6 is a schematic diagram showing training data for training the pulse condition prediction model. As shown in FIG. 6, the training data used for training the pulse condition prediction model may include pulse wave data of 11 meridians, each of the pieces of pulse wave data includes the pulse wave intensity tag and the phase difference tag and the corresponding standard deviations, and each of the pieces of training data may include label corresponding to each pulse condition. Data type and generation method of the to-be-predicted data are the same as that of the training data, only that the to-be-predicted data does not have the label corresponding to each pulse condition. Specifically, the to-be-predicted data input into the pulse condition prediction model may include the first pulse wave data of the 11 the meridians, wherein each of the pieces of first pulse wave data includes the pulse wave intensity tag and the phase difference tag and the corresponding standard deviations. The pulse condition prediction model may generate the predicted probability value of pulse condition of each to-be-predicted data. Therefore, in step S105, the processing module 12 may input the pieces of first pulse wave data of the meridians into the input layer of the pulse condition prediction model, wherein a product of the number of the meridians and the parameter number corresponding to the first pulse wave data (i.e the number of parameters corresponding to the first pulse wave data) equals to the number of neurons of the input layer. Take the example of the 11 meridians and each of the pieces of pulse wave data including the pulse wave intensity tag, the phase difference tag, the first standard deviations and the second standard deviations, the number of neurons of the input layer of the pulse condition prediction model may be 44.

Accordingly, accurate prediction results of the pulse conditions may be generated. Traditional Chinese medicine practitioners can make efficient and accurate diagnoses on the subject's health condition according to predicted probability values generated by the pulse condition prediction model and other determination results obtained by performing looking, listening, questioning and feeling. Therefore, misdiagnosis made by traditional Chinese medicine practitioners with less experience can be avoided, and the accuracy of diagnoses made by experienced traditional Chinese medicine practitioners can be further improved.

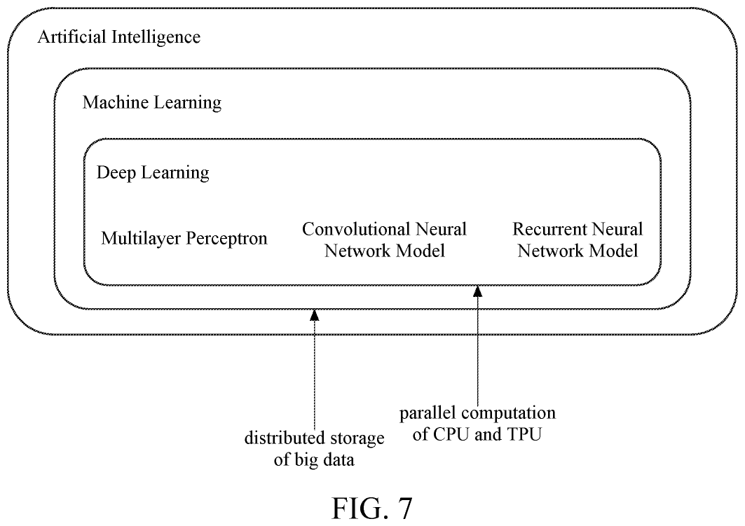

Please refer to FIG. 7, FIG. 7 is a classification structural diagram of the initial neural network model used in the present disclosure. Artificial intelligence (AI) means the computer can think like a human and perform human-like behavior. Machine learning is a branch of artificial intelligence, which means that the machine is learning through different algorithms, wherein machine learning algorithm may be used to automatically analyze and induce corresponding rule, and perform prediction on unknown data by using said rule. In addition, a large amount of training data (big data) is required during machine learning, and the training data may be stored in distributed database or file system. Deep learning is a branch of machine learning, which means that the machine performs prediction by simulating how a human neural network works. And, deep learning may be implemented by graphics processing unit (GPU) or tensor processing unit (TPU) performing parallel computation. The pulse condition prediction model described in the present disclosure may be a deep-learning model, and may include trained keras sequential model, trained multilayer perceptron (MLP), trained convolutional neural network model or trained recurrent neural network model.

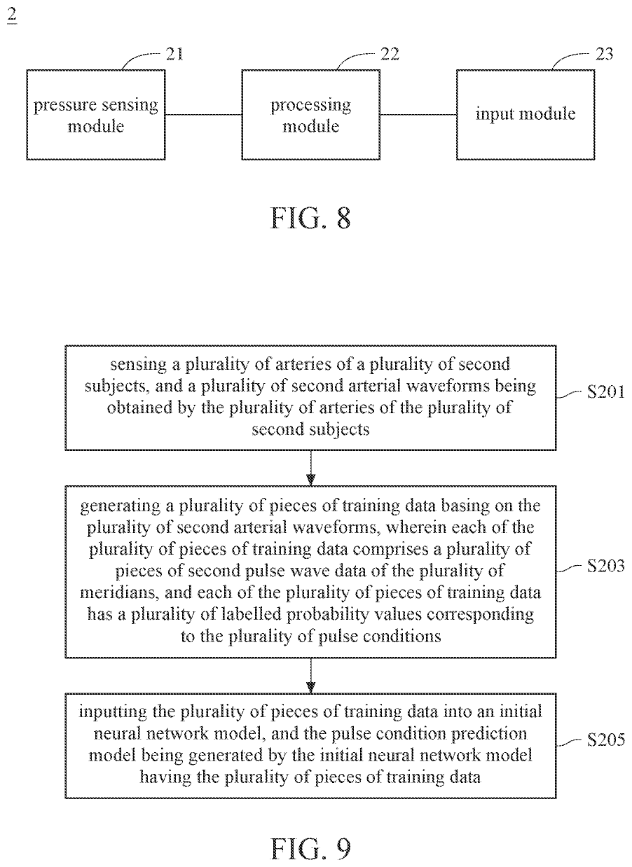

Please refer to FIG. 8, wherein FIG. 8 is a block diagram illustrating a pulse condition prediction system according to another embodiment of the present disclosure. As shown in FIG. 8, the pulse condition prediction system 2 includes a pressure sensing module 21, a processing module 22 and an input module 23. The processing module 22 is connected to the pressure sensing module 21 and the input module 23, wherein the processing module 22 may be electrically connected to the pressure sensing module 21 and the input module 23, or in communication connection with the pressure sensing module 21 and the input module 23. The implementation method of the pressure sensing module 21 and the processing module 22 of the pulse condition prediction system 2 may be the same as the pressure sensing module 11 and the processing module 12 shown in FIG. 1, their detail descriptions are not repeated herein. The processing module 22 and the processing module 12 in FIG. 1 may be the same module. That is, the processing module 22 may include one or more processors. The input module 23 may be an input device such as a mouse, a keyboard, a touch screen, a microphone etc., and the input module 23 is configured to receive labelled probability values of each pulse condition inputted by the user. The user may input the labelled probability value of each pulse condition of each of the pieces of training data through the input module 23. The labelled probability value of each pulse condition may come from experienced traditional Chinese medicine practitioner by determining the labelled probability value based on the values of the parameters of the training data.

To further explain the operation of the pulse condition prediction system 2, please refer to FIG. 8 and FIG. 9, wherein FIG. 9 is a flow chart illustrating a pulse condition prediction method according to another embodiment of the present disclosure. As shown in FIG. 9, the pulse condition prediction method may further include: step S201: sensing a plurality of arteries of a plurality of second subjects, and a plurality of second arterial waveforms being obtained by the plurality of arteries of the plurality of second subjects; step S203: generating a plurality of pieces of training data basing on the plurality of second arterial waveforms, wherein each of the plurality of pieces of training data comprises a plurality of pieces of second pulse wave data of the plurality of meridians, and each of the plurality of pieces of training data has a plurality of labelled probability values corresponding to the plurality of pulse conditions; and step S205: inputting the plurality of pieces of training data into an initial neural network model, and the pulse condition prediction model being generated by the initial neural network model having the plurality of pieces of training data. Steps shown in FIG. 9 may be performed before step S105 shown in FIG. 2.

In step S201, the pressure sensing module 21 senses the artery of each second subject to obtain the second arterial waveform. That is, a plurality of arteries of a plurality of second subjects is further sensed by the pressure sensing module 21, and a plurality of second arterial waveforms is obtained by the pressure sensing module 21. The second subject is different from the first subject. The method of the pressure sensing module 21 performing sensing to obtain the second arterial waveform may be the same as step S101 of FIG. 2, the details are not repeated herein.

In step S203, the processing module 22 generates the training data according to the second arterial waveform, wherein each of the pieces of second pulse wave data of the training data includes the pulse wave intensity tag and the phase difference tag, and the phase difference tag is the phase difference between the phase of the second arterial waveform and the phase of the default phase. In addition, each of the pieces of second pulse wave data may further include the first standard deviation corresponding to the pulse wave intensity tag and the second standard deviation corresponding to the phase difference tag. Data type of the second pulse wave data is the same as the data type of the first pulse wave data, the detail description of the second pulse wave data is not repeated herein. In addition, the processing module 22 may further receive the labelled probability value inputted by the user from the input module 23, wherein the labelled probability value represents the probabilities of the second subject having the pulse conditions.

The labelled probability values are associated with a normal condition or an abnormal condition of a respective one of the meridians, wherein the normal condition indicates that a corresponding one of the first standard deviations of the second pulse wave data or a corresponding one of the second standard deviations of the second pulse wave data is not higher than the default standard deviation, and the abnormal condition indicates that a corresponding one of the first standard deviations of the second pulse wave data or a corresponding one of the second standard deviations of the second pulse wave data is higher than the default standard deviation. For example, when a first standard deviation is greater than the corresponding default standard deviation, it means that the meridian that said first standard deviation corresponds to is in the abnormal condition. In other words, the generation of the labelled probability values of the training data of one second subject depends on the normal condition or the abnormal condition of each of the meridians of said second subject.

In step S205, the processing module 22 inputs the training data into the initial neural network model for training to generate the trained pulse condition prediction model. The initial neural network model may include keras sequential model, multilayer perceptron (MLP), convolutional neural network model or recurrent neural network model. The initial neural network model includes an input layer, a plurality of hidden layers and an output layer, wherein weighting operation and activation value of each layer affects the next layer, and the output layer outputs the final determination result. Further, value of each neuron in the input layer is multiplied with the weight of the neuron to obtain a plurality of input values, and a threshold value (referred to as a bias herein) is subtracted from a sum of the input values to determine whether the neurons are activated to further affect neurons of the next layer.

Take multilayer perceptron for example, the model can be a series model (application programming interface (API) of advanced deep learning) built on open source software database of TensorFlow (API of bottom-layer deep learning), and is developed with programming language of Python. After the model is built by using Python and the series model, the input layer, the hidden layers and the output layer may be built by using addition, and fitting method is used to start repeated training. The training result (inference) may be stored as files in HDF5 (hierarchical data format 5) format.

In multilayer perceptron, the neurons performing transmission are arranged in layers. Further, the processing module 22 uses the meridians and the parameters corresponding to the second pulse wave data as the input layer of the initial neural network model, wherein a product of the number of the meridians and the number of parameters corresponding to the second pulse wave data equals to the number of neurons of the input layer. The 11 meridians and the pulse wave intensity tag, the amplitude standard deviation, the phase difference tag and the phase standard deviation of each of the meridians (total of 44 pieces of data) may be used as the input layer of the multilayer perceptron. In other words, the input layer may have 44 neurons. The output layer may output the predicted probability value corresponding to each pulse condition. For example, assuming there are 28 pulse conditions in traditional Chinese medicine, for the 11 meridians, for one learning computation, there may be 8704 weight values (44×64+64×64+64×28=8704) need to be adjusted, and 156 bias (64+64+28=156) need to be adjusted; if the values of 10000 subjects are multiplied thereto to be used as the training data, the data amount will be too large. Therefore, the training of the initial neural network model needs to be done by computer (for example, the processing module 22).

Since each of the pieces of the training data includes the normal condition and the abnormal condition of each meridian and the labelled probability value corresponding to the training data, the trained pulse condition prediction model may generate accurate predicted probability values according to the to-be-predicted data (for example, the to-be-predicted data described in FIG. 2).

In addition to the above embodiments, after generating the pulse condition prediction model, the processing module 22 may obtain validation data, input the validation data into the pulse condition prediction model to generate a plurality of validation probability values; determine prediction accuracy of the pulse condition prediction model based on the validation probability values; and input the to-be-predicted data into the pulse condition prediction model when the prediction accuracy is higher than or equal to an accuracy threshold. The validation data may be third pulse wave data of the same data type as the first pulse wave data or the second pulse wave data, and the processing module 22 already stores known probability values corresponding to the validation data. The processing module 22 may calculate a degree of matching between the probability values generated by the pulse condition prediction model and said known probability values as the prediction accuracy. And, the processing module 22 performs step S105 of FIG. 2 when determining that the prediction accuracy is higher than or equal to the accuracy threshold.

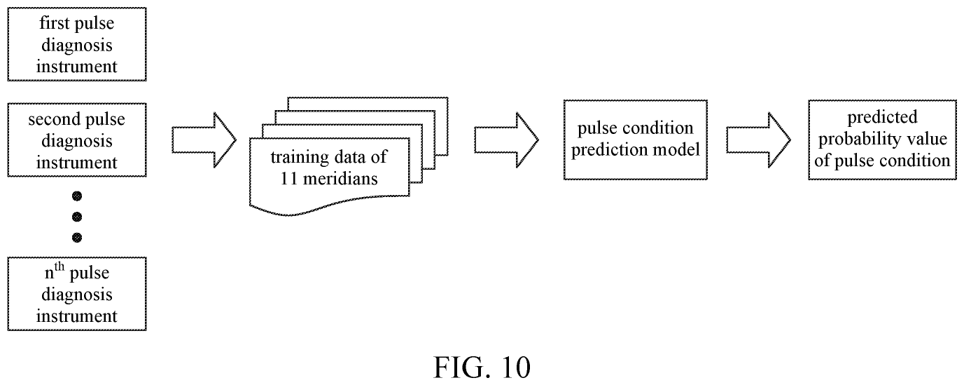

Please refer to FIG. 10, FIG. 10 is a schematic diagram illustrating model training and model application according to one or more embodiments of the present disclosure. As shown in FIG. 10, the instruments may be used to generate respective training data of the 11 meridians, each of the pieces of training data corresponds one second subject; or, one pulse diagnosis instrument may be used to generate a plurality of pieces of training data, and each of the pieces of training data corresponds one second subject. Then, the pieces of training data are inputted into the initial neural network model for training to generate the pulse condition prediction model. Then, the to-be-predicted data of the first subject may be input into the pulse condition prediction model to generate the predicted probability value corresponding to each pulse condition.

In view of the above description, the pulse condition prediction method and system according to one or more embodiments of the present disclosure may be applied to generate accurate prediction result of the pulse condition. By loading the pulse condition prediction model stored in a computer, traditional Chinese medicine practitioners can make efficient and accurate diagnoses on the subject's health condition according to predicted probability values generated by the pulse condition prediction model and other determination results obtained by performing looking, listening, questioning and feeling, and the learning curves of traditional Chinese medicine practitioners may be shortened. Therefore, misdiagnosis made by traditional Chinese medicine practitioners with less experience can be avoided, and the accuracy of diagnoses made by experienced traditional Chinese medicine practitioners can be further improved.

What is claimed is:

1. A pulse condition prediction method, comprising:
sensing, by a pressure sensing module, an artery of a first subject and a first arterial waveform being obtained by the artery of the first subject;
generating, by a processing module, to-be-predicted data basing on the first arterial waveform, wherein the to-be-predicted data comprises a plurality of pieces of first pulse wave data of a plurality of meridians; and
inputting, by the processing module, the to-be-predicted data into a pulse condition prediction model, and a plurality of predicted probability values of a plurality of pulse conditions being generated by the pulse condition prediction model having the to-be-predicted data.

2. The pulse condition prediction method according to claim 1, wherein before inputting the to-be-predicted data into the pulse condition prediction model, the method further comprises:
sensing, by the pressure sensing module, a plurality of arteries of a plurality of second subjects, and a plurality of second arterial waveforms being obtained by the plurality of arteries of the plurality of second subjects;
generating, by the processing module, a plurality of pieces of training data basing on the plurality of second arterial waveforms, wherein each of the plurality of pieces of training data comprises a plurality of pieces of second pulse wave data of the plurality of meridians, and each of the plurality of pieces of training data has a plurality of labelled probability values corresponding to the plurality of pulse conditions; and
inputting, by the processing module, the plurality of pieces of training data into an initial neural network model, and the pulse condition prediction model being generated by the initial neural network model having the plurality of pieces of training data.

3. The pulse condition prediction method according to claim 1, wherein inputting, by the processing module, the to-be-predicted data into the pulse condition prediction model comprises:
inputting the plurality of pieces of first pulse wave data of the plurality of meridians into an input layer of the pulse condition prediction model,
wherein a product of a number of the plurality of meridians and a parameter number corresponding to the plurality of pieces of first pulse wave data, equals to a number of neurons of the input layer.

4. The pulse condition prediction method according to claim 1, wherein each of the plurality of pieces of first pulse wave data comprises a pulse wave intensity tag and a phase difference tag, wherein the phase difference tag is associated with a phase difference between a phase of the first arterial waveform and a default phase.

5. The pulse condition prediction method according to claim 4, wherein each of the plurality of pieces of first pulse wave data further comprises a first standard deviation corresponding to the pulse wave intensity tag and a second standard deviation corresponding to the phase difference tag.

6. The pulse condition prediction method according to claim 2, wherein inputting, by the processing module, the plurality of pieces of training data into the initial neural network model comprises:
using the plurality of meridians and a plurality of parameters corresponding to the plurality of pieces of second pulse wave data as an input layer of the initial neural network model,
wherein a product of a number of the plurality of meridians and a parameter number of the plurality of parameters corresponding to the plurality of pieces of second pulse wave data, equals to a number of neurons of the input layer.

7. The pulse condition prediction method according to claim 2, wherein each of the plurality of pieces of second pulse wave data comprises a pulse wave intensity tag and a phase difference tag, wherein the phase difference tag is associated with a phase difference between a phase of the second arterial waveform and a default phase.

8. The pulse condition prediction method according to claim 7, wherein each of the plurality of pieces of second pulse wave data further comprises a first standard deviation corresponding to the pulse wave intensity tag and a second standard deviation corresponding to the phase difference tag.

9. The pulse condition prediction method according to claim 8, wherein the plurality of labelled probability values is associated with a normal condition or an abnormal condition of a respective one of the plurality of meridians, and the abnormal condition indicates a corresponding one of the first standard deviations or a corresponding one of the second standard deviations being greater than a corresponding default standard deviation.

10. A pulse condition prediction system, comprising:
a pressure sensing module, an artery of a first subject being sensed and a first arterial waveform being generated by the pressure sensing module; and
a processing module connected to the pressure sensing module, and to-be-predicted data being generated via the first arterial waveform by the processing module, the to-be-predicted data being inputted into a pulse condition prediction model by the processing module, and a plurality of predicted probability values of a plurality of pulse conditions being generated by the pulse condition prediction model having the to-be-predicted data,
wherein the to-be-predicted data comprises a plurality of pieces of first pulse wave data of a plurality of meridians.

11. The pulse condition prediction system according to claim 10, wherein a plurality of arteries of a plurality of second subjects is further sensed by the pressure sensing module, and a plurality of second arterial waveforms be obtained by the pressure sensing module,
wherein a plurality of pieces of training data are further generated by the processing module via the plurality of second arterial waveforms, and the plurality of pieces of training data are inputted into an initial neural network model by the processing module, and the pulse condition prediction mode is generated by the processing module,
wherein each of the plurality of pieces of training data comprises a plurality of pieces of second pulse wave data of the plurality of meridians, and each of the plurality of pieces of training data has a plurality of labelled probability values corresponding to the plurality of pulse conditions.

12. The pulse condition prediction system according to claim 11, further comprising:
an input module connected to the processing module, and the plurality of labelled probability values being received by the input module.

13. The pulse condition prediction system according to claim 10, wherein the processing module performing inputting the to-be-predicted data into the pulse condition prediction model comprises:
inputting the plurality of pieces of first pulse wave data of the plurality of meridians into an input layer of the pulse condition prediction model,
wherein a product of a number of the plurality of meridians and a parameter number corresponding to the plurality of pieces of first pulse wave data, equals to a number of neurons of the input layer.

14. The pulse condition prediction system according to claim 10, wherein each of the plurality of pieces of first pulse wave data comprises a pulse wave intensity tag and a phase difference tag, wherein the phase difference tag is associated with a phase difference between a phase of the first arterial waveform and a default phase.

15. The pulse condition prediction system according to claim 14, wherein each of the plurality of pieces of first pulse wave data further comprises a first standard deviation corresponding to the pulse wave intensity tag and a second standard deviation corresponding to the phase difference tag.

16. The pulse condition prediction system according to claim 11, wherein the processing module performing inputting the plurality of pieces of training data into the initial neural network model comprises:
using the plurality of meridians and a plurality of parameters corresponding to the plurality of pieces of second pulse wave data as an input layer of the initial neural network model,
wherein a product of a number of the plurality of meridians and a parameter number of the plurality of parameters corresponding to the plurality of pieces of second pulse wave data, equals to a number of neurons of the input layer.

17. The pulse condition prediction system according to claim 11, wherein each of the plurality of pieces of second pulse wave data comprises a pulse wave intensity tag and a phase difference tag, wherein the phase difference tag is associated with a phase difference between a phase of the second arterial waveform and a default phase.

18. The pulse condition prediction system according to claim 17, wherein each of the plurality of pieces of second pulse wave data further comprises a first standard deviation corresponding to the pulse wave intensity tag and a second standard deviation corresponding to the phase difference tag.

19. The pulse condition prediction system according to claim 18, wherein the plurality of labelled probability values is associated with a normal condition or an abnormal condition of a respective one of the plurality of meridians, and the abnormal condition indicates a corresponding one of the first standard deviations or a corresponding one of the second standard deviations being greater than a corresponding default standard deviation.

20. The pulse condition prediction system according to claim 10, wherein the a pulse diagnosis instrument is formed by the pressure sensing module and the processing module.
